(19) United States
(12) Patent Application Publication
(10) Pub. No.: US 2021/0321654 A1
(43) Pub. Date: Oct. 21, 2021

(54) High-temperature Sensitive Saccharomyces pastorianus and Application thereof

(71) Applicant: Jiangnan University, Wuxi (CN)

(72) Inventors: Qi Li, Wuxi (CN); Mingfang Zhang, Wuxi (CN); Xinyue Li, Wuxi (CN); Jinjing Wang, Wuxi (CN); Chunfeng Liu, Wuxi (CN); Chengtuo Niu, Wuxi (CN); Feiyun Zheng, Wuxi (CN)

(21) Appl. No.: 17/363,728

(22) Filed: Jun. 30, 2021

Related U.S. Application Data
(63) Continuation of application No. PCT/CN2020/095044, filed on Jun. 9, 2020.

(30) Foreign Application Priority Data
Jun. 11, 2019 (CN) 201910501260X

(51) Int. Cl. A23L 33/145; C12N 1/18; A23L 27/21

(52) U.S. Cl. CPC A23L 33/145; C12R 2001/85; A23L 27/21; C12N 1/185

(57) ABSTRACT

The disclosure relates to a strain of high-temperature sensitive S. pastorianus and application thereof and belongs to the technical field of microorganisms. In the disclosure, the S. pastorianus Pilsner is mutagenized by ARTP, based on the principle that yeast intracellular alkaline phosphatase reacts with BCIP to produce blue precipitates, screening of high-temperature sensitive mutant strains can be carried out according to the appearance time and shade of the blue, and finally a strain of high-temperature sensitive S. pastorianus P-510 is obtained after screening and quickly autolyzed at 37° C. Autolysis of P-510 at 37° C. can promote the dissolution of autolysates to obtain highly flavored nucleotides, glucan and antioxidant active substances, which can be used for production of S. pastorianus autolysates with high nutritional values.

### TECHNICAL FIELD

The disclosure relates to a strain of high-temperature sensitive S. pastorianus and application thereof and belongs to the technical field of microorganisms.

### BACKGROUND

S. pastorianus contains rich proteins (45%-60%), amino acids, B vitamins, polysaccharides, minerals and the like and is a natural source of nutritional biologically active substances. A yeast extract is a product which is obtained by degrading proteins and nucleic acid substances in yeasts according to the yeast autolysis characteristic and concentrating proteins and nucleic acid substances with other effective components into soluble nutrients, and can be used in condiments, cosmetics, health care products, medicines and other fields. Main components of a yeast extract include nucleotides, amino acids, polypeptides, B vitamins and trace elements. Since amino acids, guanylic acid (5′-GMP), inosinic acid (5′-IMP) and other components have unique flavors, a yeast extract has become an important natural flavor enhancer to replace sodium glutamate and nucleotides in processed foods. The preparation of a yeast autolysate is the same as that of a yeast extract, but subsequent high-temperature concentration is not required, which is conducive to maintaining natural structures of polypeptides, amino acids and other substances and obtaining a yeast autolysate with biological activity. Biologically active substances contained in a yeast autolysate include β-glucan, mannan, oligosaccharides, mineral ions and polypeptides, and can be used in functional foods and dietary additives. This is one of new directions in application of S. pastorianus.

### SUMMARY

The disclosure discloses a strain of S. pastorianus, has been preserved in China General Microbiological Culture Collection Center on Apr. 3, 2019, the preservation number is CGMCC NO. 17520, and the preservation address is Institute of Microbiology, Chinese Academy of Sciences, No. 3, Yard 1, Beichen West Road, Chaoyang District, Beijing.

In an embodiment, the ITS nucleotide sequence of the S. pastorianus CGMCC NO. 17520 is as set forth in SEQ ID NO.1.

The disclosure discloses a method for increasing the content of flavored amino acids in a fermented product, and the method includes adding an extract of the S. pastorianus CGMCC NO. 17520 into the fermented product; the extract is obtained by culturing and autolyzing the S. pastorianus CGMCC NO. 17520 at 37-50° C. to obtain an autolyzed solution and then extracting the autolyzed solution.

In an embodiment, the S. pastorianus CGMCC NO. 17520 is cultured at 25-30° C. for 8-16 h and then continuously cultured to stay in an autolyzed state.

In an embodiment, the S. pastorianus CGMCC NO. 17520 is cultured at 25-30° C. for 8-16 h, and cultured strain cells are collected and transferred into another culture system for autolysis at 150-200 r·min⁻¹ for 20-50 h to obtain an autolyzed solution.

In an embodiment, the mass-volume ratio of the S. pastorianus CGMCC NO. 17520 to the culture system is (50-200):1.

In an embodiment, the fermented product comprises functional foods, dietary additives and condiments.

In an embodiment, the condiments comprise soy sauces, vinegar, bean pastes, sweet soybean pastes and oyster sauces.

In an embodiment, the dietary additives comprise nutrient supplements, flavor enhancers and food processing aids.

The disclosure discloses a method for increasing the content of glucan in a yeast extract, wherein the S. pastorianus CGMCC NO. 17520 is autolyzed at 37-50° C.

In an embodiment, the S. pastorianus CGMCC NO. 17520 is cultured at 25-30° C. for 8-16 h and then continuously cultured to stay in an autolyzed state.

In an embodiment, the S. pastorianus CGMCC NO. 17520 is cultured at 25-30° C. for 8-16 h, and cultured strain cells are collected and transferred into another culture system for autolysis at 150-200 r·min⁻¹ for 20-50 h.

In an embodiment, the mass-volume ratio of the S. pastorianus to the culture system is (50-200):1.

Beneficial effects: In the disclosure, the S. pastorianus Pilsner is mutagenized by ARTP, and based on the principle that yeast intracellular alkaline phosphatase reacts with BCIP to produce blue precipitates, preliminary screening of strains can be carried out according to the appearance time and shade of the blue. At last, the RNA dissolution content of mutant strains during autolysis at 37° C. is compared, and a strain of high-temperature sensitive S. pastorianus P-510 (preservation number CGMCC NO. 17520) is obtained after screening and quickly autolyzed at 37° C. Finally, a strain of high-temperature sensitive S. pastorianus P-510 is obtained after screening and quickly autolyzed at 37° C. The flavored nucleotide content (GMP+IMP) of an autolysate obtained by autolyzing P-510 at 37° C. for 144 h is 3 times that of an original strain, after P-510 is autolyzed for 120 h, the glucan content of P-510 is 40.3% higher than that of Pilsner, and P-510 can be used for production of S. pastorianus autolysates with high nutritional values.

### Biological Material Preservation

A strain of S. pastorianus, classified as S. pastorianus, has been preserved in China General Microbiological Culture Collection Center on Apr. 3, 2019, the preservation number is CGMCC NO. 17520, and the preservation address is Institute of Microbiology, Chinese Academy of Sciences, No. 3, Yard 1, Beichen West Road, Chaoyang District, Beijing.

### BRIEF DESCRIPTION OF FIGURES

FIG. 1 shows a death rate curve of ARTP mutagenesis.

FIG. 2 shows screening of mutant strains.

FIG. 3 shows growth of P-510 at 28° C. and 37° C.

FIG. 4 shows a mortality rate curve of P-510 at 37° C.

FIG. 5 shows a nucleic acid permeability rate curve of P-510 during autolysis at 37° C.

FIG. 6 shows a protein permeability rate curve of P-510 during autolysis at 37° C.

FIG. 7 shows growth curves of Pilsner and P-510.

FIG. 8 shows the content of glucan in an autolyzed solution at 37° C.

FIG. 9 shows the DPPH scavenging rate of autolyzed solutions with the same strain concentration at 37° C.

### DETAILED DESCRIPTION

(I) Autolysis refers to a biological process in which intracellular macromolecular substances are degraded by yeast cells under the action of intracellular enzymes.

(II) An extract is a solid obtained by making an autolysate subjected to certain treatment (such as high-temperature drying and a Maillard reaction).

(III) The content of guanylic acid and inosinic acid in an autolyzed solution is determined by high performance liquid chromatography.

The content of guanylic acid and inosinic acid in an autolyzed solution is determined by high performance liquid chromatography, and chromatographic conditions include: as shown in Table 1, flow rate 0.5 mL·min⁻¹, wavelength 254 nm, chromatographic column Waters XSELECT™ HSS T3 5 μm 4.6×250 mm Column.

TABLE 1
Chromatographic conditions

| | | 0.02 mmol · L⁻¹ ammonium |
|---|---|---|
| Time/min | Methanol/% | dihydrogen phosphate/% |
| 0 | 0 | 100 |
| 10 | 5 | 95 |
| 15 | 5 | 95 |
| 15.1 | 0 | 100 |
| 25 | 0 | 100 |

(IV) A method for determining the content of glucan

The content of glucan in an autolyzed solution is determined by an aniline blue method. 1 mL of an autolyzed solution is taken and centrifuged at 12000 rpm for 5 min, and 100 μL of a supernatant is sucked and added into 1 mL of an aniline blue working solution. The mixture is subjected to a dark reaction at 50° C. and heat preservation for 30 min to form fluorescent substances. The mixture is mixed thoroughly at room temperature for 30 min, and 180 μL of the mixture is sucked into a black 96-well plate for detection. The excitation wavelength is 405 nm, the emission wavelength is 495 nm, and the fluorescence value of the reaction solution is determined. The aniline blue working solution: 12.7 mL of ultrapure water, 3.70 mL of a glycine/sodium hydroxide buffer (1.0 M glycine and 1.25 M sodium hydroxide) and 2.50 mL of a dye solution (5.0 g/L aniline blue) are mixed and stored overnight in the dark.

(V) Determination of the DPPH scavenging rate

The DPPH scavenging rate is used as an index to characterize the antioxidant ability of an autolyzed solution. A spectrophotometer method is adopted, after a 0.5072 mM DPPH free radical alcohol solution is prepared with absolute ethanol, 1 mL of the DPPH free radical alcohol solution and 1 mL of an autolyzed solution are mixed for a reaction in the dark for 30 min, and the OD₅₁₇ value is measured. A calculation formula of the DPPH scavenging rate is as follows:

$$Scavenging rate ( \% ) = ( 1 - \frac{Ai - Aj}{A 0} ) \times 100 \%$$

In the formula: A0 refers to the OD₅₁₇ value measured after mixing of the DPPH solution and absolute ethanol; Ai refers to the OD₅₁₇ value measured after a reaction of the DPPH solution and a sample; Aj refers to the OD₅₁₇ value measured after mixing and a reaction of absolute ethanol and a sample.

### Example 1: Screening of High-Temperature Sensitive S. pastorianus

(1) Selection of ARTP Mutagenesis Time

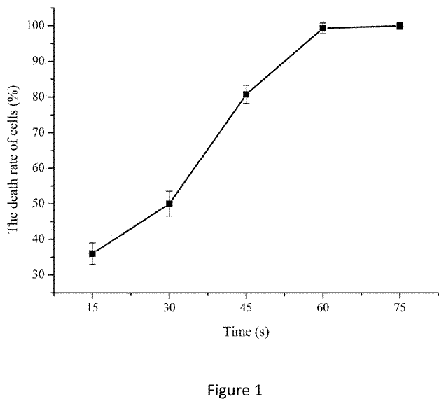

The original strain Pilsner is cultured to the logarithmic phase and diluted with normal saline until the number of cells in a strain solution is about 10⁷·mL⁻¹. The strain suspension above is subjected to ARTP mutagenesis treatment, and the treatment time is 15 s, 30 s, 45 s, 60 s and 75 s respectively. After mutagenesis is completed, treated slides are placed in 1 mL of sterile normal saline. The strain solution is reasonably diluted, spread on a YPD plate and then placed at 28° C. for culture. A death rate curve is drawn, as shown in FIG. 1. Since breeding of S. pastorianus requires maintaining the main flavor of finished beer of strains, the death rate is generally selected to be 75%-85%, and therefore, the mutagenesis time is selected to be 46 s.

(2) Screening of Mutant Strains

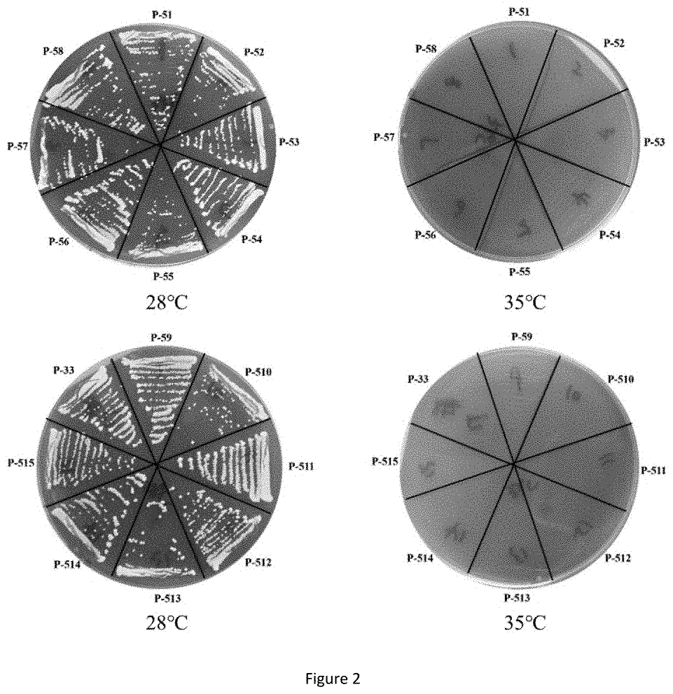

Based on the principle that yeast intracellular alkaline phosphatase reacts with BCIP to produce blue precipitates, screening of temperature sensitive mutant strains can be carried out according to the appearance time and shade of the blue. A strain solution obtained by mutagenesis for 46 s is diluted 10 times, spread on a YPD plate containing 40 mg·L⁻¹ BCIP and cultured at 28° C. for 36 h. Strain colonies are copied onto a YPD plate by a plate photocopying method and placed at 28° C. for culture, and at the same time, the YPD plate containing 40 mg·L⁻¹ BCIP is placed at 37° C. for culture for 24 h. A single strain colony with blue color is selected and cultured in 20 mL of YPD overnight, the OD₆₀₀ of the strain solution is adjusted to be 0.6-0.8, and two YPD plates are streaked at the same time and placed at 28° C. and 37° C. for culture for 24 h respectively. 16 temperature sensitive mutant strains are obtained, as shown in FIG. 2.

(3) Determination of the Autolysis Ability of Mutant Strains at 37° C.

The strains are activated overnight, centrifuged and collected, 1.0 g of a yeast paste is added into 10 mL of a citric acid buffer (pH=4.0), and the number of cells is recorded at the same time. Autolysis is carried out on a shaker at 180 r·min⁻¹ at 37° C. for 30 h, and the concentration of nucleic acid in a supernatant is measured by NanoDrop. As shown in Table 2, P-510 which is a mutant strain with high autolysis ability is obtained.

TABLE 2
The relative RNA content of an autolysate of a mutant strain at 37° C.

| | Relative RNA |
|---|---|
| Strain | content (%) |
| P-51 | 155.57 |
| P-52 | 147.72 |
| P-53 | 117.62 |
| P-54 | 140.38 |
| P-55 | 155.57 |
| P-56 | 139.93 |
| P-57 | 145.67 |
| P-58 | 139.90 |
| P-59 | 107.80 |
| P-510 | 239.51 |
| P-511 | 104.18 |
| P-512 | 111.25 |
| P-513 | 107.81 |
| P-514 | 113.22 |
| P-515 | 101.36 |
| P-33 | 160.49 |
| Pilsner | 100 |

### Example 2: Physiological Properties of High-Temperature Sensitive S. pastorianus

(1) Growth Status and Autolysis Ability of Mutant Strains at 37° C.

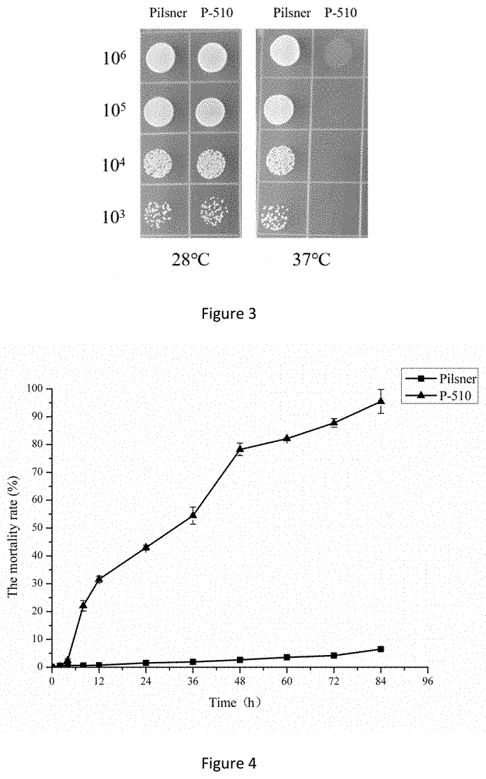

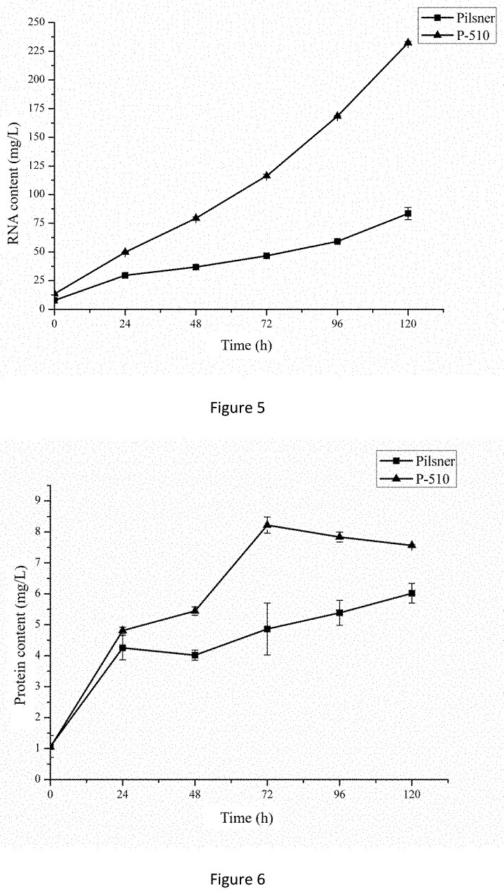

In order to further analyze the physiological properties of P-510 at 37° C., the growth status (FIG. 3) and mortality rate (FIG. 4) of P-510 at 37° C. are determined. P-510 shows significant growth inhibition at 37° C. After culture at 37° C. for 12 h, the mortality rate of Pilsner is not changed significantly and is 6.5% after 84 h, and the mortality rate of P-510 is increased rapidly and is 95.5% after 84 h, indicating that P-510 is sensitive to high temperature. The permeability rate of nucleic acid (a) and protein (b) of P-510 during autolysis at 37° C. is determined at the same time, as shown in FIG. 5. During autolysis, the content of nucleic acid and protein of an autolyzed solution is increased, and the increase of the content of nucleic acid is higher than the increase of the content of protein. After 120 h, the nucleic acid content of P-510 is 232.3 mg·L⁻¹, the nucleic acid content of Pilsner is 83.43 mg·L⁻¹, and the increase rate of the nucleic acid content of P-510 is higher than that of Pilsner and 3-3. The increase of the protein content is low, and the protein content is changed most in 0-24 h. After 120 h, the protein content of P-510 is 7.57 mg·L⁻¹, and the protein content of Pilsner is 6.02 mg·L⁻¹. In conclusion, P-510 is more autolyzed at 37° C. and more sensitive to temperature than Pilsner.

(2) Growth Curve and Fermentation Ability

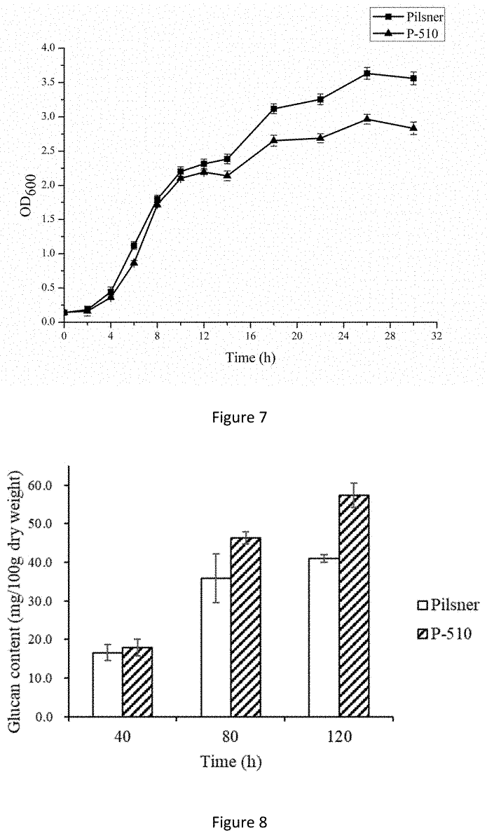

Mutagenesis may affect the growth ability and fermentation performance of strains, so that the growth curve and fermentation ability of mutant strains are determined. As shown in FIG. 7, P-510 and Pilsner enter the logarithmic phase at the same time, and the strain concentration is slightly lower than that of an original strain in a plateau, but the overall growth of strains is consistent, indicating that the growth ability of P-510 after mutagenesis treatment is not affected. At the same time, as shown in Table 3, the alcohol degree of a fermentation solution of Pilsner and P-510 after fermentation at 11° C. for 7 days is basically the same as the actual fermentation degree, indicating that normal fermentation of P-510 can be carried out.

TABLE 3
Alcohol degree and actual fermentation degree of a fermentation
solution of Pilsner and P-510

| Index | Pilsner | P-510 |
|---|---|---|
| Alcohol | 3.7 ± 0.1 | 3.62 ± 0.1 |
| degree/% v · v⁻¹ | | |
| Actual | 55.3 ± 1.0 | 54.6 ± 1.1 |
| fermentation | | |
| degree/% | | |

### Example 3: Analysis of Autolysates of High-Temperature Sensitive Mutant Strains at 37° C.

(1) Determination of the Content of Flavored Nucleotides

The high-temperature sensitive S. pastorianus P-510 screened in Example 1 is autolyzed at 37° C., and the content of guanylic acid and inosinic acid in an autolyzed solution is determined by high performance liquid chromatography. As shown in Table 4, the content of flavored nucleotides (GMP+IMP) in an autolysate obtained by autolyzing P-510 at 37° C. for 144 h is 3 times that of an original strain, indicating that high-temperature sensitive P-510 is more autolyzed at 37° C. and is beneficial to obtain a yeast extract and an autolysate with high content of flavored nucleotides.

TABLE 4
The content of GMP and IMP in autolyzed solutions of
Pilsner and P-510 with the same
strain concentration during autolysis at 37° C.

| Nucleotide | | | | |
|---|---|---|---|---|
| content | Pilsner | | P-510 | |
| (mg · L⁻¹) | 72 h | 144 h | 72 h | 144 h |
| GMP | 0.73 ± 0.05 | 0.76 ± 0.05 | 1.60 ± 0.07 | 1.81 ± 0.06 |
| IMP | 0.36 ± 0.03 | 1.04 ± 0.05 | 0.96 ± 0.06 | 3.50 ± 0.09 |
| GMP + IMP | 1.09 ± 0.08 | 1.81 ± 0.10 | 2.56 ± 0.13 | 5.31 ± 0.14 |

(2) Determination of the Content of Dextran

During autolysis, the cell wall is pressured, a variety of hydrolytic enzymes act on the cell wall, and then cell wall components including the biologically active substance β-glucan are dissolved. The content of β-glucan in autolyzed solutions of Pilsner and P-510 at 37° C. is determined separately, as shown in FIG. 3. The longer the autolysis time, the higher the content of dissolved glucan. As shown in FIG. 8, after autolysis for 120 h, the glucan content of P-510 is 40.3% higher than the glucan content of Pilsner, indicating that the content of dissolved dextran of P-510 during autolysis at 37° C. is increased due to the high-temperature sensitive mutation property.

(3) Determination of the DPPH Scavenging Rate

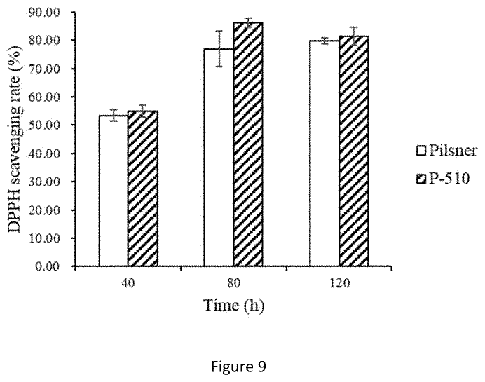

Yeast extracts and autolysates have certain antioxidant properties and can be used as functional food additives. The main reason is that active peptides degraded from proteins during autolysis of yeasts, glucan and mannan dissolved out of cell wall components and other substances have antioxidant properties. The DPPH scavenging rate of autolyzed solutions of Pilsner and P-510 at 37° C. is determined separately, as shown in FIG. 9. The highest DPPH scavenging rate of Pilsner during autolysis at 37° C. for 120 h is 79.80%, and the highest DPPH scavenging rate of P-510 during autolysis for 80 h is 86.09% and is 6.29% higher than that of Pilsner. The reason may be that when protease catalyzes hydrolysis of peptide bonds in proteins, the length of polypeptide chains, the number of free amino acids and the sequence of amino acids are affected by temperature, thereby affecting the antioxidant properties of autolyzed solutions. Therefore, it is conducive to obtaining yeast extracts and autolysates with high antioxidant properties by autolyzing P-510 at 37° C.

### Example 4: Application of High-Temperature Sensitive S. pastorianus in Preparation of a Soy Sauce

A cake is added into water and steamed; an Aspergillus oryzae strain obtained by expanded culture is inoculated into the steamed raw material, and then the high-temperature sensitive S. pastorianus of the disclosure is inoculated (or an extract of the high-temperature sensitive S. pastorianus is added) and thoroughly stirred uniformly; the inoculated koji material is ventilated for culture, the koji preparation temperature is controlled, and finished koji is prepared; salt water is added into the finished koji and stirred uniformly, the mixture is placed in a fermentation tank, the product temperature is maintained to be 42-45° C. for about 20 days, and a sauce mash is basically mature; the mature sauce mash is post-processed to obtain a post-processed soy sauce, and the post-processed soy sauce is heated, sterilized, formulated (blended), clarified and subjected to quality test to obtain a finished product which satisfies quality standards.

### Example 5: Application of High-Temperature Sensitive S. pastorianus in Preparation of Vinegar

Water is added into rice for soaking; the soaked rice is steamed; the steamed rice is heated and then quickly rinsed with cold water; Rhizopus oryzae and the high-temperature sensitive S. pastorianus of the disclosure are added into the rice (or an extract of the high-temperature sensitive S. pastorianus is added) for fermentation.

Acetic acid bacteria are inoculated into a mixture for culture; when the content of acetic acid in a fermentation system is not increased, a vinegar mash is sealed, isolated from the air and continuously fermented to obtain a mature vinegar mash; the fermented mature vinegar mash is sealed, isolated from the air, fermented at room temperature for 40-45 days and filtered with water to obtain a vinegar liquid.

### Example 6: Application of High-Temperature Sensitive S. pastorianus in Preparation of a Bean Paste

100 kg of soybeans, 4.3 kg of flour and 24.3-25.7 kg of sea salt are used as raw materials, and 0.29-0.43 kg of seed koji is added. The soybeans are steamed and uniformly mixed with the flour, the sea salt and the seed koji to prepare koji at a constant temperature of 30° C. for 24 h.

After koji is prepared, the high-temperature sensitive S. pastorianus of the disclosure is inoculated (or an extract of the high-temperature sensitive S. pastorianus is added) on the 0^{th} day when the koji is placed into a jar, and fermented at a constant temperature of 37° C. until a sauce mash is mature; the mature sauce mash is heated and sterilized to obtain a finished bean paste.

Preferred embodiments above are used to illustrate the disclosure but not to limit the disclosure. Anyone familiar with this technology can make various changes and modifications without departing from the spirit and scope of the disclosure. Therefore, the protection scope of the disclosure should be defined by the claims.

What is claimed is:

1. A recombinant strain of Saccharomyces pastorianus (S. pastorianus), which is preserved in China General Microbiological Culture Collection Center on Apr. 3, 2019, wherein the preservation number is CGMCC NO. 17520, and the preservation address is Institute of Microbiology, Chinese Academy of Sciences, No. 3, Yard 1, Beichen West Road, Chaoyang District, Beijing, China.

2. A method of using the recombinant strain of claim 1 for increasing the content of flavored amino acids in a fermented product, comprising obtaining an extract of the recombinant strain by culturing and autolyzing the recombinant strain at 37-50° C. to obtain a culture solution and then extracting the culture solution, and adding the extract into the fermented product.

3. The method according to claim 2, wherein the recombinant strain is cultured at 25-30° C. for 8-16 h and then continuously cultured to stay in an autolyzed state.

4. The method according to claim 2, wherein the recombinant strain is cultured at 25-30° C. for 8-16 h, and cultured strain cells are collected and transferred into another culture system for autolysis at 150-200 r·min⁻¹ for 20-50 h to obtain a culture solution.

5. The method according to claim 2, wherein the mass-volume ratio of the recombinant strain to the culture system is (50-200):1.

6. The method according to claim 2, wherein the fermented product comprises functional foods, dietary additives and condiments.

7. The method according to claim 6, wherein the condiments comprise soy sauces, vinegar, bean pastes, sweet soybean pastes and oyster sauces.

8. The method according to claim 6, wherein the dietary additives comprise nutrient supplements, flavor enhancers and food processing aids.

9. A method of using the recombinant strain of claim 1 for increasing the content of glucan in a yeast extract, wherein the recombinant strain is autolyzed at 37-50° C.

10. The method according to claim 9, wherein the recombinant strain is cultured at 25-30° C. for 8-16 h and then continuously cultured to stay in an autolyzed state.

11. The method according to claim 10, wherein the recombinant strain of is cultured at 25-30° C. for 8-16 h, and cultured strain cells are collected and transferred into another culture system for autolysis at 150-200 r·min⁻¹ for 20-50 h.

12. The method according to claim 9, wherein the mass-volume ratio of the recombinant strain to the culture system is (50-200):1.


SEQUENCE LISTING (1 sequence)

SEQ ID NO: 1 - DNA, 822 bp, S. pastorianus
taaggaatttatattttgaaatggatttttttttttttgttttggcaagagcatgagagcttttactgggaaagaagacaagagagggagagtccagccgggccggcgcttatgggcggggtcttgttaggtttgaaagtttctttcttgctatcccaaagggggagagatttcggggcttttgttaaaggacaattaaaaccgtttcaacacaacacactggggggttttcatatctttgcaattttttctttgggcattcgggcaatcggggcccaggggttaacaaacacaaacaattttatcttttctttaattttttgtcaaaaacaaaaattttcgaacctggaaattttaaaatattaaaaactttcaacaacggaccccttggttctcgcatcgatgaaaaacgcagcgaaatgggatacgtaatgggaattgcaaaattccgtgaatcatcgaatctttgaacgcacattgcgccccttgttattccagggggcatgcctgtttgagcgtcatttccttctcaaacattctgtttggaagggagggatactctttggagttaacttgaaattgctggccttttcattggatgttttttttttccaaagagaggtttctctgcgtgtttggggtataaggcaagtacggccgtttaaggtttaaccaactgcggttaatctttttaatactgggcgtatgggaacgttaccgaaaagaagagagcgcctgggggaacaatgttcttaaagttggacctcaactcaggaggagtccccgcgaaactaaaacatatcaataagggggggagaaaat